(19) United States
(12) Patent Application Publication
(10) Pub. No.: US 2006/0004387 A1
(43) Pub. Date: Jan. 5, 2006

(54) Ophthalmic clip and associated surgical method

(76) Inventor: Nicholas C. Caro, Glenview, IL (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(21) Appl. No.: 11/055,150

(22) Filed: Feb. 10, 2005

Related U.S. Application Data
(63) Continuation-in-part of application No. 10/882,702, filed on Jul. 1, 2004.

(51) Int. Cl. A61F 9/013

(52) U.S. Cl. 606/151; 623/4.1

(57) ABSTRACT

A method and clip for treating presbyopia and/or open angle glaucoma in which the sclera is supported or reinforced, while substantially maintaining the special relationship between the ciliary muscle and the lens. The method includes making an incision in the conjunctiva to gain access to the sclera overlying the ciliary muscle. The Tenon's capsules are moved laterally to expose the sclera, and opposed shallow pockets are made in the sclera. A clip, or series of clips, is provided having two opposed feet, which are received in the opposed pockets in the sclera. The Tenon's capsules are then slid over the clip and the conjunctiva is closed.

### CROSS REFERENCE TO RELATED APPLICATION

This application is a continuation in part of application Ser. No. 10/882,702, filed Jul. 1, 2004.

### BACKGROUND OF THE INVENTION

The present invention is directed to an ophthalmic clip for treating vision disorders, such as presbyopia and/or glaucoma and an associated surgical method for application of the clip.

Presbyopia is a vision disorder associated with aging resulting from the failure of the accommodation mechanism of the eye. The accommodative mechanism is driven principally by parasympathetic inervation of the ciliary smooth muscle. In the non-presbyopic eye, this causes the muscle to slide forward in a unified manner and produces an inward movement of the muscle. The result is a reduction in the diameter of the ciliary muscle collar that instigates a series of events leading to an ability to see near objects clearly.

Presbyopia is most frequently treated by the use of reading glasses, bifocals, and progressive multi-focal contact lenses. However, the inconveniences associated with eyeglasses and contact lenses have prompted investigation into, and the development of, surgical techniques aimed at correcting presbyopia.

Glaucoma, specifically primary open angle glaucoma, is an eye disease that progressively damages the optic nerve, thus producing certain characteristic defects in the afflicted individual's peripheral vision. Primary open angle glaucoma occurs when the eye's drainage canals become clogged over time, causing a gradual and irreversible loss of vision. It is most commonly treated with eye drops, such as PILOCARPINE, PROPINE, TIMOLOL and XALATAN, which may have side effects. Oral medications are also used.

A method for treating presbyopia and glaucoma and a scleral clip for use in the method are disclosed in my U.S. Pat. No. 6,517,555 and U.S. application Ser. No. 10/250,840, filed Mar. 5, 2003, both of which are incorporated herein by reference. The method involves applying a plurality of clips to the sclera underneath the conjunctiva. In the treatment of presbyopia, the clips serve to support or reinforce the ciliary muscles so that they may work to alter the lens diameter for focusing on close objects. In the treatment of glaucoma, the tensioning of the sclera with the clips stretches the tissues of the eye that provide for drainage, thus reducing blockage of the drainage canals and facilitating drainage of fluid from the eye.

While the clips disclosed in my above-referenced patent and application are designed for use in the methods described therein, the development process has indicated a need for improved clips that (a) are easier to apply, (b) more securely grip the sclera, and (c) have a lower profile, thus making them more comfortable to the wearer.

Thus, it the object of the invention to provide an improved clip uniquely suited for use in the treatment of presbyopia and/or glaucoma and a method for applying the clip to the eye.

### SUMMARY OF THE INVENTION

These objects, as well as others which will become apparent upon reference to the following detailed description and accompanying drawings, are accomplished by a clip for attachment to the sclera that includes a pair of opposed teeth or feet that are adapted to be received in shallow, complementarily-shaped pockets made in the sclera, thus securing the clip thereto. The clip comprises a body portion having a working length of from approximately 3.5 to 6.0 mm, a width of from approximately 1.0 to 2.5 mm, and a thickness of from 600 μm to 2.00 mm. Depending from the opposite ends of the body are feet for securing the clip to the sclera and which have a working length of approximately 200 μm. The middle portion of the body of the clip is either curved downwardly (i.e., toward a plane defined by the opposed feet) or enlarged (in thickness) with respect to the ends so that the clip, when secured to the sclera, pushes downwardly thereon to compress the sclera. In a second embodiment, the working length of the feet is between approximately 1.5 mm and 1.75 mm. In a third embodiment, an additional foot extends from each end of the body so as to overlie the feet referred to above and define a space therebetween for capturing the portion of the sclera defined by the incision for receiving the first-mentioned feet and the surface of the sclera.

The clip is formed of a resilient, biocompatible material. Preferably, the clip is made entirely of PMMA. Alternatively, the body of the clip may be made from PMMA, while the feet are made from titanium.

In a further aspect of the invention, a method for applying the clip is also provided. Pursuant to the method, the location of the ciliary muscles in the eye are determined, and an incision is made in the conjunctiva to gain access to the sclera overlying the ciliary muscles. The incision is opened to expose the sclera and opposed pockets are made in the surface of the sclera for receiving the feet of a clip, as described above. The clip is attached to the eye by introducing the feet of the clip into the pockets made in the sclera, with the downward curve of the body of the clip compressing the surface of the sclera inwardly. The conjunctiva is then closed over the clip. Optionally, a fibrin adhesive may be applied to the conjunctiva after it is closed over the clip in order to expedite the healing process.

### BRIEF DESCRIPTION OF THE DRAWINGS

FIG. 1 is a horizontal sectional view of an eyeball.

FIG. 2 is an anterior view of the eye showing the extrinsic eye muscles.

FIG. 3 is a perspective view of an improved clip in accordance with the present invention.

FIG. 4 is a front elevation of the clip of FIG. 3.

FIG. 5 is an end view of the clip of FIG. 3.

FIG. 6 is a top view of the clip of FIG. 3.

FIGS. 7-9 are similar views to FIGS. 3, 4 and 6, and illustrate a second embodiment of an ophthalmic clip according to the present invention.

FIGS. 10-12 are similar to FIGS. 3, 4 and 6, and illustrate a third embodiment of an ophthalmic clip according to the present invention.

### DETAILED DESCRIPTION

The method that utilizes the clip of the present invention is based upon the theory that the cause of presbyopia is the failure of the ciliary body to adjust the lens diameter in order to focus images onto the retina for close objects. The ciliary muscles change the lens diameter by using the sclera as support or fixation structure. As the sclera of the eye weakens due to age, the ciliary muscles lack the support needed in order to alter the lens diameter for focusing on close objects. Thus, in order to allow the ciliary muscle to alter the lens diameter to see close objects, the sclera must be supported or reinforced. Accordingly, an improved clip for reinforcing the sclera is provided, so as to form a stronger and more stable support for the ciliary muscles. The clip of the present invention accomplishes this by compressing or depressing the sclera. In effect, the sclera is strengthened, and the ciliary muscles are then able to again function properly to provide near vision.

It is believed that the method and its associated clip may also be advantageously used for the treatment of open angle glaucoma. Glaucoma, like presbyopia, is an age-related disease and is caused by a buildup of fluid pressure in the eye which damages the optic nerve. Over time, glaucoma destroys peripheral vision, thus shrinking the field of vision. In a healthy eye, the fluid produced by the ciliary tissues surrounding the lens is drained out of the eye by a series of drainage canals around the outer edge of the iris. With age, because the ciliary muscles lack support, they are less capable of maintaining these drainage canals in an open condition to allow free drainage of fluid. By supporting the sclera with the clip disclosed herein, and according to the present method, support is provided for the ciliary muscles, and the tissues of the eye that provide for drainage are stretched, thus reducing blockage of the fluid drainage canals and facilitating the drainage of fluid from the eye.

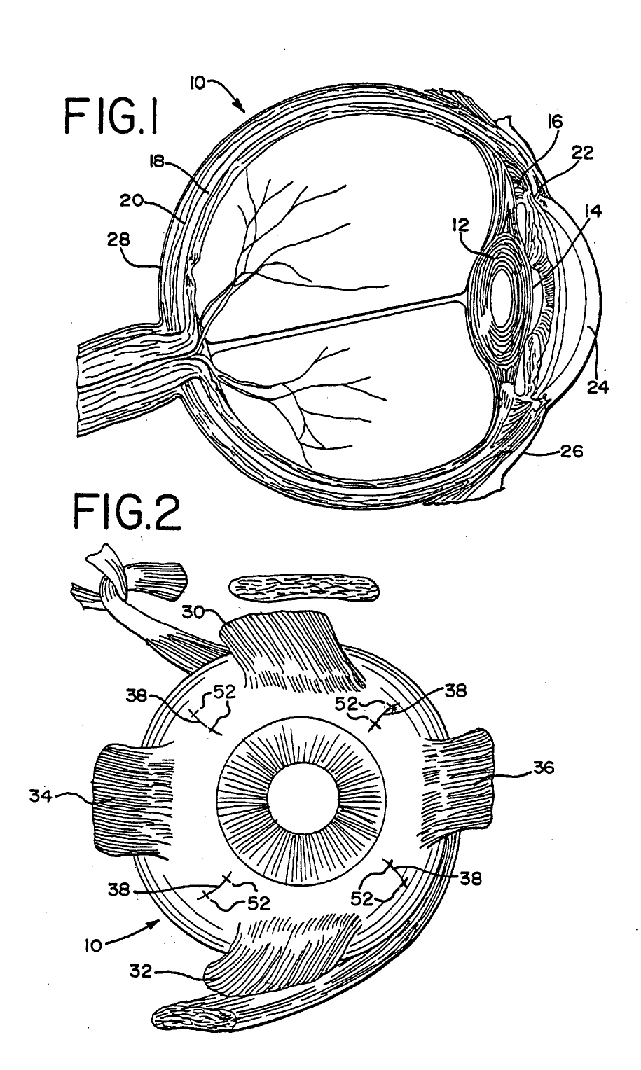

With reference to FIG. 1, there is seen a simplified sectional view of a human eye 10 having a lens 12 contained within a lens capsule 14. The ciliary body and ciliary muscle 16 are connected to the lens capsule 14 and also to the choroid 18. The sclera 20 overlies the choroid 18 and, at the front of the eye, the ciliary muscles 16, and terminates in the sclera spur 22 at the cornea 24 of the eye. The conjunctiva 26 surrounds the cornea 24 and overlies the bulbar sheath (or Tenon's capsule) 28 which, in turn, overlies the sclera 20 on the front of the eye 10. Blood is supplied to the sclera by arteries in the superior, inferior, medial and lateral rectus muscles 30, 32, 34, and 36 respectively, best seen in FIG. 2.

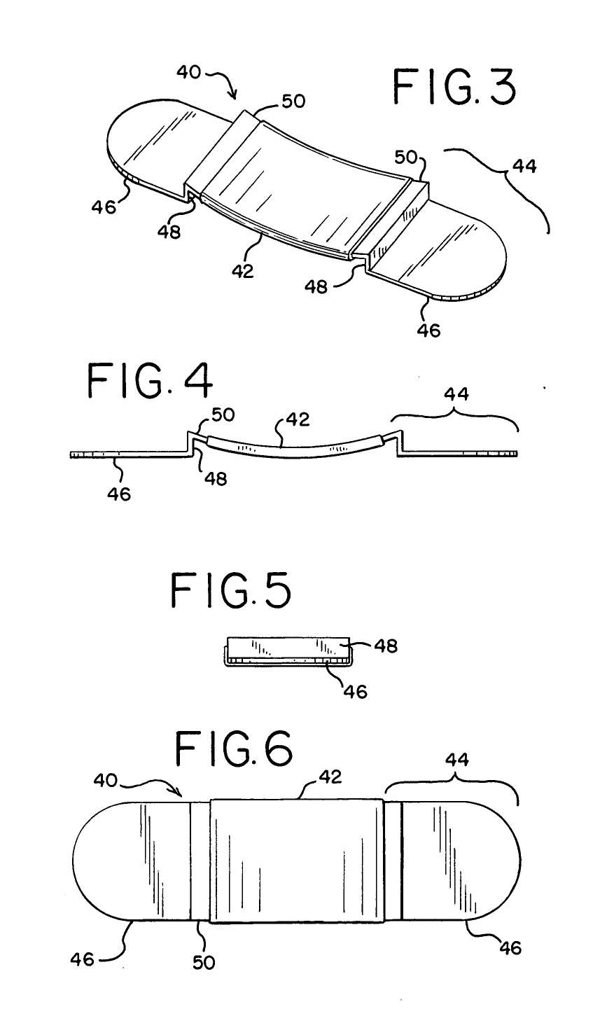

An improved clip, generally designated 40, for application sclera is shown in FIGS. 3-6. The clip, generally designated 40 includes a body portion 42, with two opposed feet 44 extending from the opposite ends of the body. As can be appreciated, the clip 40 should present no sharp edges that would irritate or damage tissue that comes into contact therewith.

In practice, the body 42 has a length that may vary from approximately 3.5 mm to 6.0 mm, depending on the desired degree of compression of the sclera. Where less compression is indicated, most likely in younger patients, the shorter clip is used. Conversely, where more compression is indicated, most likely in older patients, the longer clip is used. As can be appreciated, the length of the body 42 also generally defines the working length of the clip. The body 42 has a width of from approximately 1.0 mm to 2.5 mm, and a thickness of from between approximately 600 μm to 2.00 mm.

The opposed feet 44 are generally L-shaped (as seen in FIG. 4), with the free end of the long leg 46 of the L having a curved or semi-circular configuration (best seen in FIGS. 3 and 6) so as to reduce the likelihood of damage to tissue contacted by the feet. These curved ends are adapted to be received in pockets made in the surface of the sclera, as will be discussed in greater detail below.

The feet 44 have a working length, as defined by the long leg 46 of the L, of approximately 200 μm. The short leg 48 of the L measures between approximately 100 μm in length and 200 μm in length. Thus, the overall length of the clip 40 is the sum of the length of the two feet 44 and the length of the body 42, and consequently ranges from approximately 4.0 mm to 6.5 mm.

In keeping with an aspect of the invention, the body 42 of the clip 40 is formed with a reverse bend (i.e., the body curves downwardly) so that, when the clip 40 is applied to the eye, the clip 40 pushes down on or compresses the sclera, thus causing additional deformation of the sclera. Alternatively, the central portion of the body 42 of the clip 40 may be greater in thickness than the ends to achieve the same effect. The amount of the reverse bend is generally the same as the length of the leg 48 of the feet 44, i.e., from 100 μm to 200 μm, but may be more or less depending upon the amount of scleral compression needed.

The reverse bend exerts an inward force to assist the failing contraction of an aging ciliary body, thus providing what is known as the “Baikoff wedge effect,” named after George Baikoff, M. D. More specifically, the pressure created by the reverse bend pushes the ciliary muscle inward and forward, modifying the position of the ciliary processes and the location of the zonular plexus, and thus releasing the tension of the zonule and provoking the deformation of the crystalline lens by allowing the lens to move forward and increase its anterior curvature. This allows for an increase in the lenticular power, thus causing the lens to accommodate. Alternatively, the clip body could be enlarged in the central area between the points to provide the same effect.

The clip 40 may be made of a variety of suitable biocompatible materials, including titanium and polymethyl methacrylate (PMMA). Preferably, the entire clip is molded from PMMA. Alternatively, the body 42 of the clip 40 may be molded from PMMA, while the feet 44 are made from titanium. The titanium feet 44 are secured to the body 42 by overmolding the body with the feet 44 in situ, so that the molten PMMA flows around securement legs 50 that extend from the short leg 48 of the feet 44. The securement legs 50 may be as much as 500 μm in length, to insure that a sufficient length is received in the body 42 to maintain structural integrity. The clip may also be coated with appropriate bioactive materials, such as sytostatic drugs which have anti-inflammatory characteristics.

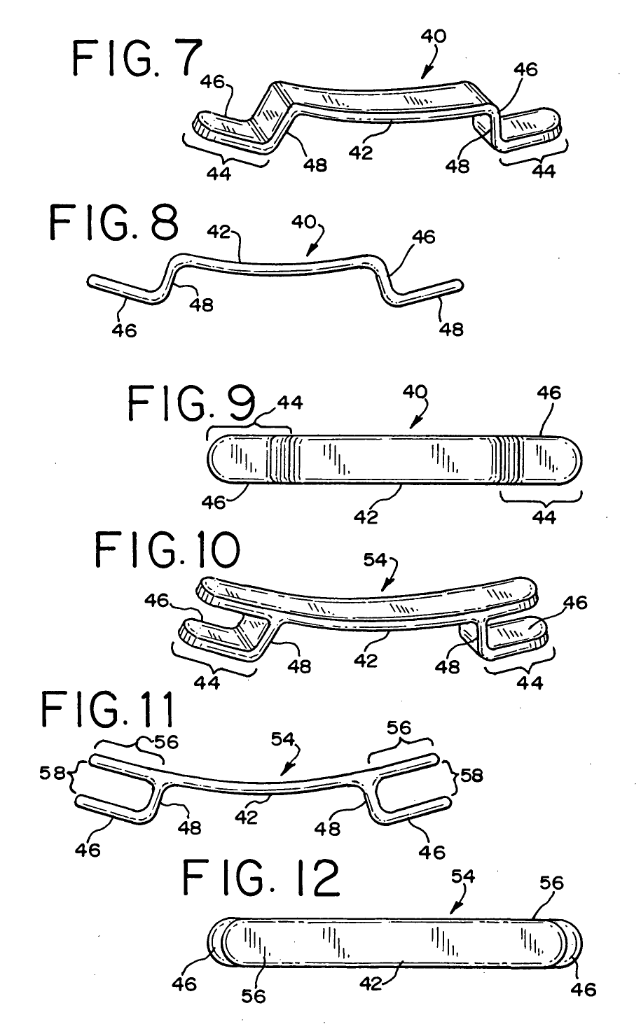

Turning to FIGS. 7-9, a further embodiment of an ophthalmic clip 40 according to the present invention is shown. The clip is similar, except dimensionally, to that shown in FIGS. 3-6, and identical reference numerals are used. With reference to FIGS. 7-9, the body 42 has a length that may vary from approximately 3.0 mm to 5.0 mm, depending on the desired degree of compression of the sclera. The clip 40 has a width of from approximately 1.0 mm to 2.5 mm, and a thickness of from between approximately 200 μm to 1.00 mm (and is preferably approximately 250 μm).

The opposed feet 44 are generally L-shaped (as seen in FIG. 8), with the free end of the long leg 46 of the L having a curved or semi-circular configuration (best seen in FIGS. 7 and 9) so as to reduce the likelihood of damage to tissue contacted by the feet. These curved ends are adapted to be received in pockets made in the surface of the sclera, as will be discussed in greater detail below.

The feet 44 have a working length, as defined by the long leg 46 of the L, of approximately 1.5 mm to 1.75 mm. The short leg 48 of the L measures between approximately 1.1 mm in length and 1.5 mm in length and is preferably approximately 1.3 mm in length. The overall length of the clip 40 ranges from approximately 5.0 mm to 7.0 mm.

The body 42 of the clip 40 is formed with a reverse bend (i.e., the body curves downwardly) so that, when the clip 40 is applied to the eye, the clip 40 pushes down on or compresses the sclera, thus causing additional deformation of the sclera. In practice the reverse bend has a radius of curvature of between 6.0 mm and about 9.0 mm and is preferably approximately 7.5 mm.

Turning to FIGS. 10-12, a third embodiment of a clip 54 according to the present invention is shown. The clip 54 is similar to that shown in FIGS. 7-9, so that identical reference numerals are used for corresponding structure. As seen in FIGS. 10-12, the clip 54 includes an additional foot 56 extending from each end of the body and overlying the foot 46 so as to define a space 58 therebetween. The space 58 is adapted to receive therein the portion of the sclera defined by the incision for making the pocket 52 for receiving the foot 46 and the surface of the sclera, and measures approximately 300-400 μm.

A method of applying the clip of the present invention to the eye will now be set forth. First, the eyelid is held open with a lid speculum and a topical anesthetic, such as a sub-conjunctival lidocaine, is applied to the eye. Then, the location of the ciliary body is determined, for example, by using commercially-available ultrasound equipment. With reference to FIG. 2, an incision 38 is then made in the conjunctiva parallel to the scleral-limbal junction so as to dissect the conjunctiva bypassing the Tenon's capsule 28. The incision is then deepened into the episclera. The incision is opened and, if necessary, the Tenon's capsule is laterally moved to expose the sclera 20. Opposed pockets 52 are made in the surface of the sclera for receiving the opposed feet of the clip using a preset marker. The openings of the pockets are spaced approximately 3.5 to 6.0 mm apart, depending on the length of the clip body, and have a depth (in a direction generally parallel to or concentric with the surface of the sclera) that corresponds to the length of the foot, i.e., from between approximately 200 μm to 1.75 mm. The pockets extend no deeper into the sclera from the surface thereof than approximately 50 percent of its thickness, i.e., no deeper than about 350 μm, and preferably extend no deeper than approximately 200 μm.

The clip is then loaded onto an application tool, which may simply comprise a grasping forceps, which grips the short legs of the feet to apply an axially compressive force to the clip along its body, thus bending the body and moving the feet toward each other. The feet are then introduced into the pockets 52 made in the sclera. If the clip 54 according to FIGS. 10-12 is used, the portion of the sclera between the incision for the pocket 52 and the surface of the sclera is received in the space 58 between in the feet 46 and 56. The applied clips have a generally low profile, closely adhering to the curvature of the eye, thus providing reinforcement to the sclera. The Tenon's capsule 28 is then reapposed over the clip and the conjunctiva closed. No suturing is needed as the conjunctiva self seals. Preferably, a fibrin adhesive, such as Tisseel® VH fibrin sealant available from Baxter Healthcare Corporation, may be applied over the closed conjunction to accelerate healing. The procedure is then repeated for each of the four quadrants, as deemed necessary by the surgeon, so that the clips are applied to the eye equally spaced about the cornea 24 between the adjacent rectus muscles. An ointment is applied to the eye, which is then patched for 24 hours.

As can be readily appreciated, the procedure can be simply reversed by merely again gaining access to the sclera by making an incision in the conjunctiva over the clip, moving the Tenon's capsule to expose the clip, and then removing the clip.

The application of each clip should deform the uvea and move the sclera inwardly approximately 0.5 mm, for a total of 2 mm if four clips are applied. This will increase the amplitude of accommodation, thus reversing the effects of presbyopia. This inward movement of the sclera should also increase the angle of the canals of Schlemn, thus increasing the aqueous flow and decreasing the intra-ocular pressure, to ameliorate the effects of glaucoma.

Thus, a method and a clip for performing the method have been provided that fully meet the objects of the present invention. While the invention has been described in terms of a preferred ophthalmic clip and method, there is no intent to limit the invention to the same. Indeed, the clip may have application to medical procedures in addition to that described above. Instead, the invention is defined by the scope of the following claims.

What is claimed:

1. An ophthalmic clip comprising an elongated body with opposed ends and opposed feet depending from each end, the body of the clip having a downward curve in a direction toward a plane defined by the opposed feet.

2. The clip of claim 1 further comprising a second foot extending from each end of the body in spaced relation to each opposed foot, the feet adapted to secure the clip to sclera tissue.

3. A medical clip for application to tissue in which the clip has an overall length of between approximately 4.0 mm and 6.5 mm and an overall width of between approximately 1.0 mm and 2.5 mm, the clip having a body with an overall length of between approximately 3.5 mm and 6.0 mm and opposed feet secured the body at opposite ends, the feet having a length of approximately 200 μm and, the body of the clip being formed with a downward curve so as to exert a force on the tissue when secured thereto.

4. The medical clip of claim 2 wherein the downward curve is from approximately 100 μm to 200 μm.

5. The medical clip of claim 2 wherein the clip is made of PMMA.

6. The medical clip of claim 2 wherein the body is made of PMMA and the feet are made of titanium.

7. The medical clip of claim 2 wherein the clip has a coating of a sytostatic drug.

8. The clip of claim 3 further comprising a second foot extending from each end of the body in spaced relation to reach opposed foot, the feet adapted to secure the clip to sclera tissue.

9. A method for treating an eye having a lens, ciliary muscles suspending the lens, sclera overlying the ciliary muscles, and conjunctiva overlying the sclera, comprising the steps of:
determining the location of the ciliary muscles;
making an incision in the conjunctiva to gain access to the sclera overlying the ciliary muscle;
opening the incision to expose the sclera;
providing a clip comprising an elongated body having opposed ends with feet depending from each end, the body of the clip having a downward curve;
making opposed pockets in the exposed surface of the sclera for receiving the feet of the clip;
attaching the clip to the eye by introducing the feet of the clip into the pockets made in the sclera, the downward curve of the body of the clip compressing the surface of the sclera inwardly; and
closing the conjunctiva over the clip.

10. The method of claim 9 wherein the opposed pockets are made to have openings spaced apart from approximately 3.5 mm to approximately 6.0 mm, the pockets extending through the sclera a depth of less than approximately 350 μm.

11. The method of claim 10 wherein the pockets extend through the sclera a depth of less than approximately 200 μm.

12. The method of claim 9 further comprising applying a fibrin adhesive to the conjunctiva after it is closed over the clip.

13. A medical clip for application to tissue in which the clip has an overall length of between approximately 5.0 mm and 7.0 mm and an overall width of between approximately 1.0 mm and 2.5 mm, the clip having a body with an overall length of between approximately 3.0 mm and 5.0 mm and opposed feet secured the body at opposite ends, the body of the clip being formed with a downward curve so as to exert a force on the tissue when secured thereto.

14. The clip of claim 13 further comprising a second foot extending from each end of the body in spaced relation to reach opposed foot, the feet adapted to secure the clip to sclera tissue.

15. The medical clip of claim 11 wherein the downward curve has a radius of curvature of from approximately 6.0 mm to 9.0 mm.

16. The medical clip of claim 11 wherein the clip is made of PMMA.

17. The medical clip of claim 11 wherein the clip has a coating of a sytostatic drug.
